(19) United States
(12) Patent Application Publication
(10) Pub. No.: US 2007/0204356 A1
(43) Pub. Date: Aug. 30, 2007

(54) PiggyBac constructs in vertebrates

(75) Inventor: Malcolm J. Fraser, Granger, IN (US)

Correspondence Address: JAGTIANI + GUTTAG, 10363-A DEMOCRACY LANE, FAIRFAX, VA, 22030, US

(73) Assignee: University of Notre Dame du Lac, Notre Dame, IN (US)

(21) Appl. No.: 11/711,025

(22) Filed: Feb. 27, 2007

Related U.S. Application Data
(60) Provisional application No. 60/776,920, filed on Feb. 28, 2006.

(51) Int. Cl. A01K 67/027; C12N 15/09

(52) U.S. Cl. 800/20; 435/325; 435/455

(57) ABSTRACT

The piggyBac transposon is disclosed herein as an extremely versatile helper-dependent vector for gene transfer and germ line transformation in a wide range of vertebrate species. Presented are methods wherein genome sequencing databases may be examined using piggyBac, as homologues of piggyBac have been found among several sequenced animal genomes, including the human genome. This transposon is demonstrated to provide transposition in primate cells and embryos of the zebra fish, Danio rerio. PiggyBac mobility is demonstrated using an interplasmid transposition assay that consistently predicts the germ line transformation capabilities of this mobile element in several species. Both transfected COS-7 primate cells and injected zebrafish embryos supported the helper-dependent movement of tagged piggyBac element between plasmids in a cut-and-paste, TTAA target-site specific manner. The present invention discloses the use of piggyBac as a tool for genetic analysis of vertebrates.

### CROSS-REFERENCE TO RELATED APPLICATIONS

This application makes reference to co-pending U.S. Provisional Patent Application No. 60/776,920, entitled “INTERPLASMID TRANSPOSITION DEMONSTRATES PIGGYBAC MOBILITY IN VERTEBRATE SPECIES” filed Feb. 28, 2006, the entire disclosure and contents of which are hereby incorporated by reference.

### GOVERNMENT INTEREST STATEMENT

This research was supported by NIH grants RO1 AI033656 and RO1 AI48561.

### BACKGROUND

1. Field of the Invention

The present invention relates generally to the field of genetic tools useful in the analysis and manipulation of vertebrate species. The invention also relates to the field of methods for using a piggyBac construct, as methods for using a piggyBac transposon in a vertebrate system, are presented.

2. Related Art

The Lepidopteran-derived piggyBac transposon is the type element for a unique group of TTAA-targeting Class II transposable elements originally isolated as mutation-inducing insertions in baculovirus genomes (Fraser et al., 1983; Fraser et al., 1985; Cary et al., 1989; Wang et al., 1989; see Fraser, 2001 for a review). Initial functional analyses confirmed its potential as a helper-dependent gene transfer vector (Fraser et al., 1995), and subsequent demonstrations of its effectiveness as a gene transfer vector have been performed in a number of invertebrate species including the important disease vectors Aedes aegypti (Kokoza et al., 2001, Lobo et al., 2002) and Anopheles gambiae (Grossman et al., 2001; Kim, et al., 2004). Its range of utility has been expanded into non-arthropod invertebrates such as Planaria (Gonzalez-Estevez, et al., 2003). As yet there has been no demonstrated mobilization of piggyBac in prokaryotic organisms.

This unique piggyBac transposon has also become established as a highly useful transgenic vector for the model genetic system, Drosophila melanogaster (Bonin and Mann, 2004; Hacker et al., 2003; Horn et al., 2003; Handler and Harrell, 1999; Parks et al., 2004; Ryder and Russell, 2003; Lorenzen et al., 2003; Thibault et al., 2004). By using piggyBac in conjunction with P-element as an insertional mutagenesis tool in Drosophila, the number of genes tagged in mutational screens has been significantly expanded (Parks et al., 2004; Thibault et al., 2004). In applying this vector to this invertebrate species, there has been a demonstrated potential for a wide variety of useful genetic manipulations (Parks et al., 2004; Thibault et al., 2004).

Plasmid based transposition assays (Lobo et al., 1999; 2001; Coates et al., 1995, 1997, Sarkar et al., 1997; Thibault et al., 1999) have provided some evidence for pursuing a given transposon as a gene transfer tool in a given species. These assays have been used to predict the capabilities for germ-line transgenesis of Sleeping Beauty in a variety of vertebrate systems, and the Tol2 element in zebrafish (Ivics et al., 1997; Kawakami et al., 1998; Izsvak et al., 2000; Kawakami et al., 2000).

In the case of the piggyBac element, the interplasmid transposition assay allows detection of precise insertion and excision events (Elick et al., 1996, 1997; Lobo et al., 1999), a defining feature of the transpositional movement of this element (Fraser et al., 1995; Fraser et al., 1996; Elick et al., 1996). In every case, demonstration of interplasmid mobilization of piggyBac sequences in non-vertebrate cells or embryos of a given species has led to successful transgenic manipulation of that species (Lobo et al., 1999; Lobo et al., 2002; Grossman et al., 2000; Grossman et al., 2001).

The most successful transgenesis system currently available for vertebrates is the pantropic retrovirus vector (Lin et al., 1994; Gaiano et al., 1996a, b; Amsterdam and Hopkins, 1999). Pantropic retrovirus vectors provide a significant improvement in the identification of mutated genes compared to chemical mutagenesis strategies by tagging genes associated with a phenotypic alteration (Gaiano et al., 1996b; Amsterdam and Hopkins, 1999). In addition, the retrovirus approach potentially allows reinsertion of mutated genes for analysis of function, limited promoter or enhancer trapping, or directed gene knockouts using RNAi approaches (e.g. Sablitzky et al., 1993; Korn et al., 1992; Xiong et al., 1999). However, retrovirus vectors lack some significant capabilities of an ideal transgenesis vector. By way of example, these deficiencies include remobilization following insertion and a carrying capacity greater than 10 kb. These retroviral vectors are also difficult to produce and present a biohazard to laboratory personnel (Linney et al., 1999; BD Biosciences/Clontech manual). A suitable transposon vector would provide a more desirable alternative to retroviruses in developing functional genomics of vertebrate systems.

A more suitable transposon vector for use in vertebrates should facilitate the identification of tagged genes through frequent and mechanistically predictable insertion and excision, as well as allow defined regulation of movement permitting the development of enhancer and suppressor trapping capabilities. These manipulations are essential for full development of functional genomics in vertebrates.

A need continues to exist in the genetic arts for improved, precise insertion, excision, and remobilization techniques and tools to achieve saturation mutagenesis, enhancer genetic trapping, and non-viral transgenic engineering in vertebrate systems. Such techniques and tools would also satisfy the long felt need for accomplishing detailed genetic analyses and engineering of vertebrate systems.

### SUMMARY

In a general and overall sense, the present invention provides molecular tools and methods for using these molecular tools in the mobilization, characterization, manipulation and transformation a vertebrate genome, utilizing a piggyBac transposon element. In particular, these methods may be used in the genetic manipulation of vertebrates, including primates, such as humans. The methods and constructs may be further described as providing very site specific and predictable techniques for producing specifically engineered genetic products of interest.

Methods and Assays:

In one aspect, the invention provides an interplasmid assay for vertebrate cells and tissues that includes a piggyBac transposon element.

In another aspect, the invention provides a piggyBac transposon mobilization method for vertebrates. In some embodiments, the method comprises preparing a piggyBac donor plasmid comprising a piggyBac transposon, combining a defined ratio of piggyBac donor plasmid, a target plasmid and a helper with a vertebrate nucleic acid of interest to provide a piggyBac interplasmid transposition product, and providing a modified vertebrate nucleic acid sequence having therein a mobilized vertebrate nucleic acid sequence of interest, wherein said helper comprises a piggyBac transposase.

In another aspect, a method is provided comprising a genetic mobilization method that employs an interplasmid transposition assay format. According to some embodiments, the method comprises formation of an interplasmid transposition product (IPT), the IPT comprising a piggyBac transposon element. In some embodiments, the piggyBac transposon element includes a detectable tagging element, such as an identifiable molecular tag. By way of example, the molecular tag may comprise a drug resistance gene, such as an antibiotic resistant gene. One such example is a kanamycin resistance gene.

In some embodiments, the genetic mobilization method may be described as a helper-plasmid dependent genetic mobilization method. In some embodiments, the helper-plasmid is further described as comprising a nucleic acid sequence encoding a transposase, an enzyme that is capable of cutting out a piece of nucleic acid (DNA) and moving it to a different place.

In some of these particular embodiments, the helper and/or helper moiety comprises a helper plasmid, such as a phspBac plasmid or pBKOα plasmid. In particular embodiments, the helper plasmid comprises a mammalian promoter region or a viral promoter region (such as a CMV promoter). In some embodiments, the helper moiety is a transcribed RNA encoding a piggyBac transposase.

In some embodiments the target comprises a target plasmid, such as pGDV1.

In some embodiments, the donor comprises a donor plasmid, such as pB(KOα).

In another broad aspect, a method is provided for mobilizing a desired segment or piece of nucleic acid of interest in a fertilized embryo or cell. This genetic mobilization method may be used with all types of vertebrate cells and organisms. By way of example, the nucleic acid sequences and selected segments thereof within an embryo, such as the zebrafish embryo, and within a cell, such as a primate cell (including but not limited to a human cell), may be modified according to the methods described herein. By way of example, a primate human cell line in which the piggyBac mobilization method may be used is a human kidney cell line, such as the COS-7 cell line.

In particular aspects, the mobilization method may be described as comprising preparing a piggyBac donor plasmid comprising a piggyBac transposon, combining a piggyBac donor plasmid, a target plasmid and a helper moiety with a vertebrate nucleic acid of interest to provide a piggyBac interplasmid transposition product (IPT), and providing a modified vertebrate nucleic acid sequence having therein a mobilized vertebrate nucleic acid sequence of interest, wherein said helper moiety comprises a nucleic acid sequence encoding a piggyBac transposase. In some embodiments, the vertebrate nucleic acid is derived from a primate cell, such as a human cell or a COS-7 primate cell.

In some embodiments, the target:donor;helper moiety is provided to a culture of cells, such as vertebrate cells, in a defined ratio. By way of example, the defined ration may be a ratio of 2:1:1.

In some embodiments, the frequency of transformation of primate cells is about 3.0×10⁻⁴ to about 6.0×10⁻⁴.

In some embodiments, the method may be described as a TTAA-site directed mobilization method.

Several advantages are presented with the present methods and assays. One of these advantages includes the ability to effectively and precisely move larger segments of nucleic acid in a vertebrate genome than had previously been possible. For example, the methods and piggyBac constructs described herein are suitable for mobilizing and analyzing nucleic acid moieties comprising a desired nucleic acid sequence that has a molecular weight of about 10 kb or greater. By way of example, the mobilization method and constructs described herein may be described as providing a vehicle for moving fragments of nucleic acid of between about 10 kb to about 300 kb, or about 15 kb to about 200 kb or about 15 kb to about 150 kb. In some embodiments, the mobilization may be described as providing for the mobilization of about 15 kb of a nucleic acid sequence of interest without any significant loss of efficiency, or even with an about 100% efficiency.

In other embodiments, the genetic mobilization method may be described as a vertebrate germ cell line transformation method.

In another general aspect, a method for modifying a vertebrate nucleic acid sequence is provided.

In yet another broad aspect, a method for mapping and/or otherwise charting and characterizing a vertebrate genome is provided, again using the piggyBac transposon. In some embodiments, the method comprises characterizing a desired region of interest in a vertebrate genome comprising the steps of mobilizing a desired region of interest of a vertebrate cell nucleic acid sequence as defined herein, wherein said desired region comprises a detectable genetic tag and a piggyBac vector sequence, to provide a transformed vertebrate nucleic acid comprising a tagged nucleic acid sequence of interest, extracting the transformed vertebrate nucleic acid and selecting the tagged nucleic acid sequence of interest, and characterizing the tagged nucleic acid sequence of interest within the transformed vertebrate nucleic acid sequence. In some embodiments, the vertebrate genome of interest is a human genome.

PiggyBac Transposon Constructs, Interplasmid Transposition Product Constructs:

According, and in a first broad aspect of the present invention, there is provided a piggyBac transposon construct suitable for use in the genetic manipulation of a vertebrate genome.

By way of example, the piggyBac transposon construct in some embodiments may be described as comprising a piggyBac transposon sequence. In some embodiments, the piggyBac transposon comprises an interplasmid transposition product transposon depicted in FIG. 1. In some embodiments, the interplasmid transposon comprises a construct having a structure as depicted for pBKOα in FIG. 1.

In some embodiments, the transposon vector comprises a vertebrate nucleic acid moiety comprising an identifiable vertebrate nucleic acid moiety of interest, a piggyBac transposon nucleic acid sequence and a transposase enzyme encoding nucleic acid sequence. In some embodiments, the vertebrate nucleic acid sequence comprises a primate cell nucleic acid moiety of interest. In other embodiments, the vertebrate nucleic acid moeity comprises a recoverable detectable molecular marker.

According to a second broad aspect of the invention, there is provided an interplasmid transposition product comprising the construct depicted at FIG. 1 (see last panel). The following abbreviations are used throughout the description of the present invention:

COS-7—a vertebrate cell line of African green monkey kidney cells (primates).

IPT—interplasmid transposition product;

REN—Restriction endonclease;

Transposon vector—a plasmid containing the piggyBac transposon or minimal sequence of the piggyBac transposon within which sequences may be inserted and thereby mobilized within cells of vertebrate or eukaryotic species.

### BRIEF DESCRIPTION OF THE DRAWINGS

The invention will be described in conjunction with the accompanying drawing, in which:

FIG. 1, according to some embodiments of the invention, presents a combination of 3 plasmids that were introduced into cells or embryos. The donor plasmid, pB(KOα), carries a piggyBac element marked with the kanamycin resistance gene, ColE1 origin of replication (ori), and the α peptide of the β-galactosidase gene. The transposase providing helper plasmid, phspBac expressed the piggyBac ORF under the control of the D. melanogaster hsp70 promoter and is unable to transpose as it lacks a terminal repeat. The target B. subtilis plasmid, pGDV1, is incapable of replication in E. coli, and contains the chloramphenicol resistance gene. Transposition of the genetically tagged piggyBac element from the donor into the target plasmid pGDV1 with the help of the transposase provided by the helper phspBac, results in an interplasmid transposition (IPT) product. This pGDV1 derived IPT plasmid with its acquired ColE1 ori can replicate in E. coli and produce blue colonies on LB/kan/cam/X-gal plates. Blue colonies that grew on LB/kan/cam/X-gal plates were grown up and plasmid DNA isolated for sequencing to confirm piggyBac mediated transposition.

### DETAILED DESCRIPTION

The mobility of piggyBac demonstrated in the present disclosure in a variety of model systems and target organisms permits the testing, verification, and perfection of strategies in easily manipulated models, and application of those proven strategies to other, less tractable models. Based upon these observations, the piggyBac element may also be used to mediate germ line transformation in many higher vertebrates, extending its effective range throughout the animal kingdom.

The extension of piggyBac mobility into vertebrate systems is useful and innovative from a genetic and functional genomic standpoint. In addition, this transposon in particular, and virtually any transposon like piggyBac with vertebrate homologues, may also be used for applied genetic engineering of agricultural or medical pest species according to the present invention. Post-transformation inactivation of a piggyBac transposon also provides an additional advantage of the invention.

### Description

It is advantageous to define several terms before describing the invention. It should be appreciated that the following definitions are used throughout this application.

Definitions

Where the definition of terms departs from the commonly used meaning of the term, applicant intends to utilize the definitions provided below, unless specifically indicated.

For the purposes of the present invention, the term “transpose” means to move, omit (delete), add, duplicate, invert, rearrange, or otherwise change the location or character of a desired nucleic acid segment.

For the purposes of the present invention, the term “transposition” means the movement of the transposon or any transposon-encompassed or bounded nucleic acid sequence from one integration site to another integration site using the transposase in either a “cis” or “trans” expressed manner.

For the purposes of the present invention, the term “helper plasmid moiety” is defined as an expression plasmid that transcribes a functional transposase RNA that in turn translates into a functional transposase protein which operates in transposition. Such transcription may be in vivo, within cells or tissues of the organism of concern, or may be ex-vivo in a test tube to be applied by transfection or injection with the donor nucleic acid moiety.

For the purposes of the present invention, the term “donor nucleic acid moiety” is defined as the transposase or transposon-bounded nucleic acid to be mobilized.

### EXAMPLES

The description of the present invention is enhanced by the various examples that follow.

### Example 1

### Methods

The present example provides a description of the materials and methods employed in the practice of the present invention.

Preparation of Plasmid DNAs:

Plasmid DNAs used for transfections or microinjections were prepared using the rapid boiling procedure and were purified by CsCl gradient centrifugation. Following collection of the supercoiled fraction and extraction of the ethidium bromide with isoamyl alcohol, the DNAs were dialyzed against four changes of 4000 volumes of 0.1×SSC and stored frozen at −20° C. until used. Because these plasmids were to be used for transfection of cell cultures they were handled as sterile reagents at all times. At no time were these DNAs subject to contamination with any other plasmids.

The target plasmid used in these analyses was pGDV1, a chloramphenicol resistance plasmid derived from the Bacillus subtilis plasmid pTZ12 (Aoki et al., 1987) by the addition of a multiple cloning site between 1970 and 2029 bp (Bron, 1995; Sarkar et al., 1997). The pB(KOα) plasmid (Thibault et al., 1999) was used as the piggyBac donor and was derived from a p3E1.2 plasmid derivative by insertion of a cartridge containing the kanamycin resistance gene, the ColE1 origin, and the α peptide of β-galactosidase at a unique BglII site within the piggyBac open reading frame. The transposase helper was the phspBac (formerly named pBhsΔSac) expression plasmid (Handler et al., 1998).

A stock plasmid mixture of pGDV1 (0.8 μg/μl), pB(KOα) (0.4 μg/μl) and phspBac (0.4 μg/μl) was prepared in sterile distilled water and used for all of the COS-7 and zebrafish experiments. A separate stock plasmid mixture of pGDV1 (0.8 μg/μl) and pB(KOα) (0.4 μg/μl) was used for the transfection and injection controls.

Maintenance and Transfection of COS-7 Cells:

African green monkey kidney cells (COS-7) were maintained by passage at 1:5 dilutions in a 37° C. incubator 5% CO2 in DMEM medium (Life Technologies) with 10% Fetal Bovine Serum (Life Technologies).

Transfection:

Transfections were performed using a starting cell density of 5×10⁴ cells/well of a 6-well culture plate. The LipofectAMINE Plus Reagent (Life Technologies, Inc.) was combined with a total of 10 μg of the stock experimental plasmid mixture in and added to COS-7 monolayers according to the manufacturer's recommended procedure. Control transfections utilized the same reagents and 10 ug of the stock control plasmid mixture containing pGDV1 and pB(KOα) to verify both a lack of contaminating positive transposition plasmids among these reagents, and the absence of endogenous piggyBac transposase activity in COS-7 cells.

Microinjection of Danio rerio Embryos:

Fresh zebrafish eggs were collected (Friemann Centre, University of Notre Dame) and injected with DNA solution as described by Westerfield (1993). Microinjection of plasmid DNA was carried out using an agarose gel (made in Hanks Solution, Westerfield, 1993) with depressions, created by a capillary tube, as a holding place (Westerfield, 1993). The DNA solution was air-pressure-injected approximately an hour after fertilization at the 1-8 cell stage zebrafish. Injected eggs were stored at 28° C. in Hanks Solution for 18 hours.

Each injection set was performed independently of the others using the same plasmid DNA preparations. The experimental injections used the stock plasmid mixture of pGDV1, pB(KOα), and (phspBac). The control injections utilized the stock control plasmid mixture containing pGDV1 and pB(KOα) to verify both a lack of contaminating positive transposition plasmids and the absence of endogenous piggyBac transposase activity in the zebrafish embryos.

Plasmid Excision Assay:

A standard transposon plasmid excision assay (Lobo et al., 1999) was performed to determine if the COS-7 primate cells could support the first step of the cut-and-paste reaction mediated by the piggyBac transposase. This assay utilized the transposase helper plasmid, phspBac, to drive excision of the piggyBac element from the donor plasmid, pBKOα. Equal concentrations of both plasmid DNAs were transfected at 2 and 5 ug total DNA concentration, recovered by modified Hirt (1967) extraction (Lobo et al., 1999; 2001) at 24 hours, digested with BglII, electroporated into DH10B cells, and immediately plated without recovery on LB/Amp/X-gal plates. No heat shock was used to induce expression of the transposase from the helper.

Because excision of the transposon results in removal of the lacZ gene in the plasmid, positive excision events are recovered as white colonies on LB Amp/Xgal plates. The number of recoverable donor plasmids for each experiment was estimated by electroporating a 1 ul aliquot of undigested DNA from the same Hirt extract and counting the number of blue colonies representing the donor plasmid.

Interplasmid Transposition Assay:

Plasmid DNAs were recovered from COS-7 cells at 24 hours post transfection, and from zebrafish embryos at 18 hours post injection using a modified Hirt (1967) extraction (Lobo et al., 1999; 2001), and electroporated into E. coli DH10B cells. Neither cells nor embryos were subjected to heat shock to induce transposase expression from the helper plasmid. Interplasmid transposition events were identified and characterized by immediate selective plating of electroporated bacteria on LB Chloramphenicol (Cam; 25 μg/ml)/Kanamycin (Kan; 50 μg/ml)/X-gal (0.025 μg/ml) plates essentially as previously described (Lobo et al., 1999; 2001)). The total amount of donor plasmid recovered was estimated by simultaneous plating of an aliquot (1%) of the transformation mix on LB Ampicillin (50 μg/ml)/X-gal (0.025 μg/ml) plates and recording the estimated number of blue colonies.

Control transfections or injections were performed using the donor and target plasmids in the absence of the helper phspBac element insuring both that no endogenous transposase activity is evident in either COS-7 cells or zebrafish embryos. In addition, a control transformation of E. coli with the stock experimental plasmid mix containing all three plasmids verified the absence of background transposition events occurring in the transformed bacteria and confirmed the absence of contaminating positive transposition plasmids among all three plasmid reagents.

### Example 2

### Interplasmid Transposition Products with PiggyBac

The present example demonstrates the preparation of an interplasmid transposition product (IPT) with the piggyBac element and the characterization of insertion sites in a target plasmid.

Method:

Plasmids were recovered by Hirt extraction 24 hours following transfection of COS-7 cells and were transformed into E. coli DH10B cells. One percent of the transformed cells were plated without recovery on LB/amphicillin plates with X-Gal, and the number of blue colonies containing donor plasmids (pB(KOα)) was counted or, where necessary, estimated (# donor plasmid). The remaining cells were plated without recovery on LB plates containing Cam, Kan, and X-Gal, and blue colonies resulting from transposition events into the target plasmid (pGDV1) were counted and sequenced using the piggyBac-specific inverse primers JFO1 and JFO2 (Methods) to determine the number of precise Interplasmid Transposition events (#IPT events). The frequency of transposition into the target pGDV1 plasmid was calculated relative to the estimated number of donor plasmids recovered. Control transfections consisted of cells transfected with donor and target plasmids alone. An additional control to demonstrate a lack of bacterial mobilization and absence of contaminating transposition plasmids consisted of the three plasmids directly transformed into E. coli DHR10B cells.

Results:

The results from this study are presented in Table 1.

As demonstrated in the results of table 1, a frequency of transformation of primate cells, COS-7 cells, of 5.7×10⁻⁴ was achieved using the piggyBac IPT method.

TABLE 1
Transposition of piggyBac in the COS-7 Cell line

| Cell | Helper | | # wells per | # Donor | # IPT | |
|---|---|---|---|---|---|---|
| transformed | plasmid | Extraction | Extraction | plasmid | events | Frequency |
| COS-7 | phspBac | 1 | 6 | 19,800 | 5 | 5.7 × 10⁻⁴ |
| | | 2 | 1 | 800 | 3 | |
| | | 3 | 1 | 1,800 | 0 | |
| | | 4 | 1 | 300 | 3 | |
| | | 5 | 1 | 300 | 3 | |
| | | 6 | 1 | 3,100 | 1 | |
| | | 7 | 1 | 100 | 0 | |
| | | 8 | 1 | 100 | 0 | |
| | | Total | 13 | 26,300 | 15 | |

Controls:

| | | | | | | |
|---|---|---|---|---|---|---|
| COS-7 | None | 1 | 12 | ˜700,000 | 0 | 0 |
| | | 2 | 12 | ˜700,000 | 0 | |
| | | 3 | 1 | 1,000 | 0 | |
| | | 4 | 1 | 900 | 0 | |
| | | Total | 26 | ˜1,401,000 | 0 | |
| E. coli | phspBac | 13 | — | ˜127,000 | 0 | 0 |

### Example 3

### Transposition of PiggyBac in Zebrafish, D. rerio

The present example demonstrates the results achieved in D. rerio embryos (Zebrafish) using a piggyBac transposon element.

Method:

Plasmids were recovered by Hirt extraction 18 hours following microinjection of zebrafish embryos. One percent of the transformed cells were plated without recovery on LB/ampicillin plates with X-Gal, and the number of blue colonies containing donor plasmids (pB)KOα)) was counted or, where necessary, estimated (# donor plasmid). In several of the control injections, the number of donor plasmids was estimated to be approximately the same the remaining cells were plated without recovery on LB plates containing Cam, Kan, and X-Gal, and blue colonies resulting from transposition events into the target plasmid (pGDV1) were counted and sequenced using the piggyBac-specific inverse primers JF01 and JF02 (Methods) to determine the number of precise Interplasmid Transposition events (# IPT events). The frequency of transposition into the target pGDV1 plasmid was calculated relative to the counted or estimated number of donor plasmids recovered. As controls, embryos were injected with the donor and target plasmids in the absence of the helper plasmid (phspBac), and the three plasmids were transformed directly into E. coli DH10B cells.

Results:

The results from this study are presented in Table 2.

TABLE 2
Transposition of piggyBac in D. rerio embryos

| | Helper | | # | # Donor | # IPT | |
|---|---|---|---|---|---|---|
| Experimental | plasmid | Injection | eggs injected | plasmids | events | Frequency |
| Zebrafish | phspBac | 1 | 110 | 1,401,000 | 1 | 1.4 × 10⁻⁶ |
| | | 2 | 350 | 1,418,400 | 1 | |
| | | 3 | 150 | 26,000 | 1 | |
| | | 4 | 400 | 367,150 | 4 | |
| | | 5 | 300 | ˜3,000,000 | 3 | |
| | | Total | 1310 | ˜7,116,100 | 8 | |

Controls:

| | | | | | | |
|---|---|---|---|---|---|---|
| Zebrafish | None | 1 | 200 | ˜1,500,000 | 0 | 0 |
| | | 2 | 300 | ˜1,500,000 | 0 | |
| | | 3 | 200 | ˜1,500,000 | 0 | |
| | | 4 | 200 | ˜1,500,000 | 0 | |
| | | Total | 900 | ˜6,000,000 | 0 | |
| E. coli | phspBac | 1 | — | 56,000 | 0 | 0 |
| | | 2 | — | 71,000 | 0 | |
| | | Total | — | 127,000 | 0 | |

### Example 4

### Interplasmid Transposition Assay in COS-7 Cells Using DGDV1Δ148 as Target

The present example demonstrates the utility of the piggyBac construct in COS-7 cells.

Method:

Plasmids were recovered by Hirt extraction 24 hours following transfection of COS-7 cells and the DNAs obtained were transformed into E. coli DH10B cells. One percent of the transformed cells was plated without recovery on LB/ampicillin plates with X-Gal, and the number of blue colonies, indicating the number of donor plasmids (pB(KOα)), was determined (# donor plasmid). The remaining cells were plated without recovery on LB plates containing Cam, Kan, and X-Gal, and the number of blue colonies, indicating transposition events into the target plasmid (pGDV1Δ148), were counted and sequenced using the piggyBac-specific inverse primers JF01 and JF02 (Methods) to determine the number of precise Interplasmid Transposition events (# IPT events). Control transfections consisted of cells transfected with donor and target plasmids alone. The frequency of transposition into the target pGDV1Δ148 plasmid was calculated relative to the number of donor plasmids recovered.

Results:

The results from these studies are presented in Table 3.

TABLE 3
Interplasmid Transposition Assay in COS-7
Cells Using pGDV1Δ148 as Target

| | Helper | | # wells in | # Donor | # IPT | |
|---|---|---|---|---|---|---|
| | plasmid | Expt | Expt. | plasmid | events | Frequency |
| Cell | | | | | | |
| trans- | | | | | | |
| formed | | | | | | |
| Cos-7 | phspBac | 1 | 1 | 336,800 | 12 | 3.56 × 10⁻⁵ |
| | | 2 | 1 | 420,000 | 14 | 3.33 × 10⁻⁵ |
| Control | | | | | | |
| Cos-7 | None | 1 | 1 | 468,300 | 0 | 0 |
| | | 2 | 1 | 542,800 | 0 | 0 |

### Example 5

### Excision Assay for PiggyBac Mobilization in Vertebrate Cells

The present example demonstrates the utility of the piggyBac transposon as a predictable tool for manipulation of selected pieces of vertebrate nucleic acid.

Because the piggyBac transposon moves using a precise cut-and-paste mechanism (Elick et al., 1996; Lobo et al., 1999, 2001), a plasmid excision assay can be used as a predictor of piggyBac transposase activity. A standard excision assay (Lobo et al., 2001) was performed in COS-7 cells and zebrafish embryos using the donor and helper plasmids, pBKOα and phspBac, respectively. The transposase providing helper plasmid, phspBac (Handler et al., 1998) expresses the piggyBac ORF under the control of the D. melanogaster hsp70 promoter, which has a demonstrated activity in vertebrate cells (Romano et al., 2001).

In both systems excision events were uncovered in the presence of the phspBac helper that were exclusively precise, characteristic of piggyBac transposase activity (Elick et al., 1996), while no excision events were recovered in the absence of the helper. These results demonstrated the activity of the piggyBac transposase in mediating the first step of the cut-and-paste movement of the element, and confirmed the utility of the Drosophila hsp70 promoter in these systems to drive expression of the transposase gene.

### Example 6

### Primate Cell-Line Transposition

The present example demonstrates the utility of the piggyBac element in transforming a line of primate cells.

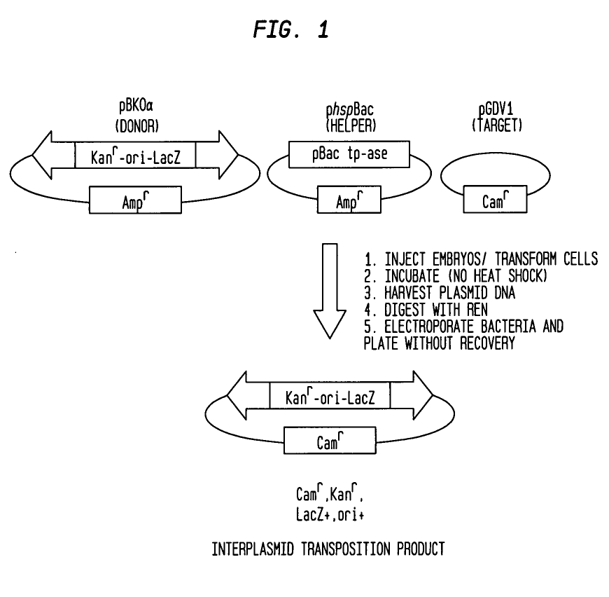

An interplasmid transposition assay (Thibault et al., 1999; Lobo et al., 2001; FIG. 1) was utilized to demonstrate that the piggyBac element was capable of helper dependent transposition in vertebrate cells. The assay is an accurate predictor of germ-line transposition and measures the ability of the piggyBac element to move from a donor plasmid (pB(KOα)) into a target plasmid (pGDV1) in the presence of piggyBac transposase expressed from the helper plasmid (phspBac).

COS-7 cells, a vertebrate cell line derived from African green monkey kidneys (Gluzman et al., 1981) were co-transfected with a combination of these three plasmids. Positive transposition events were recovered from Hirt extracts of transfected COS-7 cells by plating transformed bacteria on Cam/Kan/X-gal plates. No transposition events were recovered from control transfections in the absence of the helper plasmid, demonstrating the recovered transpositions were not the result of endogenous transposase activity and the lack of contaminating positive plasmids in the donor and target plasmid preparations. A further standard control in these assays transformed all three plasmids directly into E. coli (Table 1). Since it had previously been determined that there is no piggyBac mobility in these bacteria, this control effectively establishes the absence of contaminating positive plasmids among the three starting plasmid preparations.

Transposition frequencies were estimated relative to the total number of recovered donor plasmids, Amp/X-gal plates (Table 1). Fifteen interplasmid transposition events were recovered in 8 independently performed transfections, yielding a calculated cumulative interplasmid transposition frequency of 5.7×10-4 (Table 1).

All putative interplasmid clones were sequenced using the JF01 or JF02 outward-facing piggyBac specific primers (Methods), allowing identification of the insertion site on the pGDV1 plasmid. Confirmation of a transposition event was obtained by observing the characteristic duplication of a TTAA target site in the pGDV1 sequence on each side of the inserted transposon. Transposition events were recovered at only one of the 21 available TTAA target sites that do not result in an interruption of the chloramphenicol resistance gene (between 1169 and 1655 bp) in the pGDV1 plasmid, at base pair position 363 (Table 2), and all insertions at this site were in the same orientation.

### Example 7

### PiggyBac Interplasmid Transposition Assay in Vertebrate Study Model

The present example demonstrates the utility of the present invention in a widely used vertebrate animal model, the zebrafish.

Following the successful demonstration of transposition in a mammalian cell line, piggyBac movement was tested in the phylogenetically distant and experimentally valuable vertebrate model, the zebrafish (Danio rerio). Due to its prolific reproduction and the external development of a transparent embryo, the zebrafish is a prime model for genetic and developmental studies, as well as research in toxicology and genomics. The vertebrate zebrafish has comparable organs and tissues to the human, such as heart, kidney, pancreas, bones and cartilage. Therefore, demonstration of piggyBac transposase activity in the zebrafish here demonstrates the utility of the present invention for the identification, characterization and manipulation of genetic material in these and other organs and tissues in the human with the piggyBac transposition technique.

Zebrafish embryos were injected at the 1 to 8 cell stage with a 2:1:1 ratio of target:donor:helper plasmid ratio in a total concentration of 1.6 μg/μl. Plasmid DNA was recovered from the injected embryos 18 hours post injection by Hirt extraction, electroporated into E. coli and assayed on selective media as described for the for COS-7 cells.

Plasmid DNAs recovered from blue Kan/Cam colonies were sequenced to verify the transpositional insertion of the KOα-marked piggyBac element into the pGDV1 target plasmid. A total of 10 interplasmid transposition events were recovered from 5 independent injection experiments yielding a combined total of 1310 injected embryos, and resulting in a cumulative interplasmid transposition frequency of 1.4×10⁻⁶ (Table 2). All clones possessed the characteristic TTAA tetranucleotide target site duplication flanking the inserted transposon which confirms piggyBac-mediated transposition. All recovered insertions occurred at base pair position 363 in the plasmid pGDV1, and all were in the same orientation. Control transformations of the combined plasmids in E. coli yielded no interplasmid transposition events (Table 2), confirming a lack of contaminating positives, and that the observed mobility was occurring in the zebrafish embryos and not in subsequently transformed bacteria.

### Example 8

### Contamination Studies

The present example is provided to demonstrate the utility of the invention as providing a genetic mobilization method employing the piggyBac transposon that is relatively free of any contaminating and/or unrelated insertional events present in the mixtures of products and/or plasmids.

Contamination is ruled out as between the present investigators studies for several reasons. First, the plasmid mixtures for each series, whether COS-7 or zebrafish, were also used in several control transfections, injections, or transformations that demonstrated both a lack of mobility due to resident piggyBac homologues known to be present in each species genome (Sarkar et al., 2004) and a lack of contaminating positive plasmids in all the reagents used, including the pGDV1, helper, and donor plasmids.

If contamination of reagents were a problem, positive insertion events would have been recovered in one or more of these controls. Likewise, if starting plasmids were contaminated in some manner, then transposition events would have been recovered in one or more of the controls, and at the very least, in the direct transformations of the plasmids in E. coli. Second, each of the transfection or injection studies were carried out independently and at separate times, with all Hirt extraction and bacterial transformation reagents having been freshly prepared. In addition, the electroporation competent E. coli DH10B were commercially prepared, and therefore free of contamination from manipulation.

### Example 9

### PiggyBac TTAA Target Site Preference—Interplasmid Transposition Assay Using a Deletion Mutation of PGDV1

In previous analyses of a number of independent insertion sites (Li et al., 2005), it was established that there was no apparent consensus sequence configuration apart from the TTAA target site necessary for insertion of the piggyBac transposon. However, these interplasmid transposition results demonstrate a preferential insertion of the piggyBac vector at the TTAA target site at 363 bp among all alternative sites in the pGDV1 plasmid.

Since contamination had been ruled out as a factor, an experimental verification of some alternative explanation for the observed target site preference was conducted.

Preferential insertion at a given position in the pGDV1 plasmid was contemplated as the result of factors other than sequence recognition. Therefore, a spontaneous deletion mutation of pGDV1, named pGDV1Δ148, which has a deletion of sequence between 506 and 654 bp., was used. Utilizing pGDV1Δ148 as the target plasmid in an interplasmid transposition assay in COS-7 cells, 25 of 26 individual insertions were recovered at the TTAA site at position 85 bp (Table 3) instead of position 363 bp. Simultaneous control transfections in the absence of the helper plasmid yielded no transformation events. All these insertions were in the same orientation as those previously observed at position 363 bp. These results demonstrate that the previously observed preferential insertions at 363 bp likely result from a plasmid configuration effect rather than the affinity for a specific sequence.

piggyBac transposition in cells of two vertebrate species is provided. Utilizing a previously established excision and interplasmid transposition assays, the piggyBac element can mobilize in both the COS-7 vertebrate cell line and in fertilized zebrafish embryos. In both cases mobility is absent in the absence of the piggyBac transposase demonstrating that the intact piggyBac transposase is necessary for transposition and endogenous piggyBac homologues do not provide a detectable level of independent transposition events. As observed in a previous report (Lobo et al., 1999) mobility is not detected when the plasmids are passaged through E. coli demonstrating that the eukaryote intracellular environment is necessary for transposition and the prokaryotic intracellular environment is apparently unfavorable.

The frequency of transposition observed in zebrafish embryos is two orders of magnitude less than the frequency typically obtained with this assay in insect embryos as well as the frequency obtained in this study for interplasmid transposition in COS-7 cells, possibly reflecting the relative inefficiency of the Drosophila heat shock promoter in expressing the transposase in zebrafish. This frequency is similar to those frequencies obtained with other transposable elements in zebrafish embryo injections. This relatively consistent reduced frequency observed among all transposons applied in injected zebrafish embryos could reflect an inherently unfavorable environment for unprotected DNA.

In these transposition assays of both COS-7 and zebrafish embryos, all observed insertions were limited to one of the 21 recoverable TTAA insertion sites that do not interrupt the chloramphenicol gene on the target pGDV1 plasmid, and all were in the same orientation. This target site and orientation preference is also observed in non-vertebrate cells. For example, others have reported an apparent preference for insertion at one or two target sites within pGDV1 in both D. melanogaster and A. aegypti (Lobo et al., 1999). In these insect embryos, insertions were limited to positions 363 and 491, with position 363 being the most favored site in Drosophila. Further, the insertions recovered at position 393 in all insect species previously tested happened to be in the same orientation (Lobo et al., 1999), corresponding with the orientation observed in the present studies. In contrast, insertions at several alternative sites were recovered from the lepidopterans Trichoplusia ni (Lobo et al., 1999) and Pectinophora gossypiella (Thibault et al., 1999), with no apparent orientation preferences at those alternative sites.

While initial studies in some species indicated a species-dependent preference for certain TTAA sites, subsequent analyses proved these apparent preferences were not strict. Therefore, it is believed that target site preference may be the result of limited sampling size.

The pGDV1 plasmid may present a configuration within some cells that favors insertion at a particular TTAA target site, and possibly a particular orientation at that site. This interpretation is further supported by a second interplasmid transposition assay performed in COS-7 cells using the pGDV1Δ148 deletion plasmid which removed 148 bp of sequence between 506 and 654 of the pGDV1. Using this plasmid as the target, no insertions were recovered at position 393, however 25 of 26 individual insertions were recovered at position 85, all in same orientation. This orientation corresponded to the orientations observed for all position 85 insertions recovered in previous insect embryo assays (Lobo et al., 1999), and for those position 393 insertions recovered in the present COS-7 and zebrafish assays.

### Example 10

### PiggyBac Interplasmid Transposition Product

The present example is presented to demonstrate the utility of a composition of matter described herein as a piggyBac interplasmid transposition product, as a tool for examining and modifying genomes in virtually any desired target, including plants, insects, plasmids, and any prokaryotic or eukaryotic genome or piece of isolated nucleic acid from or in a cell, tissue, or whole organism.

Using the teachings of the present disclosure in primate cells, and the disclosure in prior work of the present inventor in non-vertebrates, it is anticipated that the present interplasmid transposition product method may be used in the mobilization and/or modification of the genome of a plant, protest, invertebrate or vertebrate, without an undue amount of experimental trial and error, given ordinary skill in the art. The teachings of Ding et al. (2005), Cell, 22:473-483, Wilson et al. (2007), Molecular Therapy, 15(1): 139-145, Cedric Feschotte, (2006), PNAS, 103(41): 14981-14982, and Thomas Bestor (2005), Cell, 122:322-325 are specifically incorporated herein in their entirety for the purposes of providing additional supplemental technical teaching to be used in conjunction with the teachings herein in the practice of these additional embodiments of the invention.

All documents, patents, journal articles and other materials cited in the present application are hereby incorporated by reference.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart there from.

### BIBLIOGRAPHY

The references listed below as well as all references cited in the specification are incorporated herein by reference to the extent that they supplement, explain, provide a background for or teach a methodology, techniques, and/or compositions employed herein.
1. Aoki, T., et al. (1987), Gene, 51:107-111.
2. Amsterdam, A. and Hopkins, N. (1999), Methods in Cell Biology, 60:87-98.
3. Bonin, C. P. and Mann, R. S. (2004), Genetics, 167:1801-1811.
4. Bron, R. (1995) Plasmids. Molecular biological methods for Bacillus, pp. 75-174, edited by C. R. Harwood and S. M. Cutting, John Wiley and Sons, New York.
5. Cary, L. C., et al. (1989). Virology, 172:156-169.
6. Coates, C. J., et al. (1997), Mol Gen Genet., 253:728-733.
7. Coates, C. J., et al. (1995), Mol Gen Genet., 249:246-252.
8. Ding S, et al. (2005), Cell., 122:473-83.
9. Elick, T. A., Bauser, C. A. and Fraser, M. J. (1996), Genetica 98:33-41.
10. Elick, T. A., Lobo, N. and Fraser, M. J. (1997), Mol Gen Genet, 255:605-610.
11 Fraser, M. J. (2001) The TTAA-Specific Family of Transposable Elements: Identification, Functional Characterization, and Utility for Transformation of Insects. Insect Transgenesis: Methods and Applications. A. M. Handler and A. A. James. Boca Raton, Fla., CRC Press: 249-268.
12. Fraser, M. J., et al. (1985), Virology, 145:356-361.
13. Fraser, M. J., et al. (1995), Virology, 211:397-407.
14. Fraser, M. J., et al. (1996), Insect Mol. Biol., 5:141-151.
15. Fraser, M. J., et al. (1983), J. Virology, 47:287-300.
16. Gaiano, N., (1996a), Proc. Natl. Acad. Sci, 93:7777-7782.
17. Gaiano, N., et al. (1996b), Nature 383:829-832.
18. Gluzman, Y. (1981) Cell, 23:175-182.
19. Gonzalez-Estevez, C., et al. (2003) Proc. Natl. Acad. Sci. 100 (24):14046-14051.
20. Grossman, G. L., et al. (2001), Insect Mol. Biol., 10:597-604.
21. Grossman, G. L., et al. (2000), Insect Biochem. Mol. Biol., 30:909-914.
22. Hacker, U., et al., (2003), Proc Natl Acad Sci, 100:7720-7725.
23. Handler, A. M., Harrell, R. A., (1999), Insect Mol. Biol. 8:449-57.
24. Handler, A. M., et al. (1998), Proc Natl Acad Sci 95:7520-7525.
25. Handler, A. M., et al., (2004), Nat Biotechnol, 22(9):1150-1154.
26. Hirt, B. (1967), J Mol. Biol., 26:365-369.
27. Horn, C., et al. (2003), Genetics, 163:647-661.
28. Ivics, Z., et al. (1997), Cell, 91:501-510.
29. Izsvak, Z, Ivics, Z. and Plasterk, R. H. (2000), J. Mol. Biol., 302:93-102.
30. Kawakami, K., et al. (1998), Gene, 225:17-22.
31. Kawakami, K., et al. (2000), PNAS 97(21):11403-11408.
32. Kim, W., et al. (2004), J Med Entomol, 41(3):447-455.
33. Kokoza, V., et al. (2001), Insect Biochem Mol. Biol., 31:1137-1143.
34. Korn, R., et al. (1992), Mech. Dev., 39:95-109.
35. Li, X, et al. (2001), Mol Genet Genomics, 266:190-198.
36. Li, X., et al. (2005), Insect Mol. Biol., 14:17-30.
37. Lin, S., et al. (1994), Science, 265:666-669.
38. Linney, E., et al. (1999), Developmental Biology, 213:207-216.
39. Lobo, N. F., et al. (2002), Insect Mol. Biol., 11:133-139.
40. Lobo, N., et al. (1999), Mol Gen Genet., 261:803-810.
41. Lobo, N., et al., (2001), Mol Genet Genomics, 265:66-71.
42. Lorenzen, M. D., et al. (2003), Insect Molecular Biology, 12 (5):433-440.
43. Luo, G., et al. (1998), Proc. Natl. Acad. Sci. 95:10769-10773.
44. Parks, A. L., et al. (2004), Nat Genet, 36 (3):288-292.
45. Romano, G., et al. (2001), Gene Ther. 8:600-607.
46. Ryder, E. and Russell, S. (2003), Transposable elements as tools for genomics and genetics in Drosophila. Brief Funct Genomic Proteomic 2:57-71.
47. Sablitzky, F., et al. (1993), Cell Growth & Differentiation, 4:451-459.
48. Sarkar, A., et al. (1997), Genetica 99:15-29.
49. Sarkar, A., et al. (2004), Mol Genet Genomics, 270:173-180.
50. Thibault, S. T., et al. (1999), Insect Mol. Biol. 8:119-123.
51. Thibault, S. T., et al. (2004), Nat. Genet. 36:283-287.
52. Wang, H. H., et al. (1989), Gene 81:97-108.
53. Westerfield, M. (1993), The Zebrafish Book. University of Oregon Press, Eugene, Oreg.
54. Xiong, J.-W., et al. (1999) Retroviral promoter-trap insertion into a novel mammalian septin gene expressed during mouse neuronal development. Mechanisms of Development 86:183-191.

What is claimed is:

1. A piggyBac transposon mobilization method for vertebrates comprising:
preparing a piggyBac donor plasmid comprising a piggyBac transposon;
combining a piggyBac donor moiety, a target moiety and a helper moiety with a vertebrate nucleic acid of interest to provide a piggyBac interplasmid transposition product; and
providing a modified vertebrate nucleic acid sequence having therein a mobilized vertebrate nucleic acid sequence of interest,
wherein said helper moiety comprises a nucleic acid sequence encoding a piggyBac transposase.

2. The method of claim 1 wherein the vertebrate nucleic acid is derived from a primate cell.

3. The method of claim 2 wherein the primate cell is a COS-7 primate cell.

4. The method of claim 3 wherein the helper comprises a helper moiety comprises a helper plasmid having a vertebrate or viral promoter region.

5. The method of claim 4 wherein the helper plasmid comprises a viral promoter region.

6. The method of claim 3 wherein a frequency of transformation of primate cells is about 3.0×10⁻⁴ to about 6.0×10⁻⁴.

7. The method of claim 1 wherein the vertebrate is a zebrafish.

8. The method of claim 4 wherein the helper comprises a helper plasmid phspBac.

9. The method of claim 1 wherein a defined ratio of target:donor:helper is a combined with the nucleic acid of said vertebrate cell.

10. The method of claim 9 wherein the target:donor:helper is provided to a culture of zebrafish at a ratio of 2:1:1.

11. The method of claim 1 wherein the helper moiety is a transcribed RNA encoding a piggyBac transposase.

12. The method of claim 9 wherein a total DNA concentration in the combining step is 1.6 μg/ul, wherein the total DNA concentration comprises the donor moiety, target moiety and helper moiety nucleic acid, or comprising the donor moiety and helper moiety nucleic acid.

13. The method of claim 1 wherein the target comprises a target plasmid pGDV1.

14. The method of claim 1 wherein the donor moiety comprises a donor plasmid pB(KOα).

15. The method of claim 1 wherein the helper moiety comprises a sequence encoding a transposase.

16. The piggyBac mobilization method of claim 1 further defined as a TTAA-site directed mobilization method.

17. The method of claim 1 wherein the nucleic acid moiety mobilized in the vertebrate genome is 10 kb or greater in size.

18. A method for characterizing a desired region of interest in a vertebrate genome comprising:
mobilizing a desired region of a vertebrate cell nucleic acid sequence according to the method of claim 1, wherein said desired region comprises a detectable genetic tag and a piggyBac vector sequence, to provide a transformed vertebrate nucleic acid comprising a tagged nucleic acid sequence of interest;
extracting the transformed vertebrate nucleic acid and selecting the tagged nucleic acid sequence of interest; and
characterizing the tagged nucleic acid sequence of interest within the transformed vertebrate nucleic acid sequence.

19. The method of claim 18 wherein the vertebrate genome of interest is a human genome.

20. A piggyBac interplasmid transposition product comprising an identifiable vertebrate nucleic acid moiety of interest, a piggyBac transposon nucleic acid sequence and a transposase enzyme encoding nucleic acid sequence.

21. The piggyBac interplasmid transposition product of claim 20 comprising a primate cell nucleic acid moiety of interest.

22. The piggyBac interplasmid transposition product of claim 20 wherein the vertebrate nucleic acid moiety comprises a recoverable detectable molecular marker.
